(19) United States
(12) Patent Application Publication
(10) Pub. No.: US 2010/0185259 A1
(43) Pub. Date: Jul. 22, 2010

(54) GARMENT FOR ELECTRICALLY STIMULATING MUSCLES

(75) Inventors: Naoto Shiba, Kurume-shi (JP); Hitomi Yamaguchi, Kurume-shi (JP); Yoshihiko Tagawa, Kitakyushu-shi (JP); Kiyoshi Numada, Oyabe-shi (JP); Kazuhiro Yoshikawa, Tenri-shi (JP); Keiji Murakami, Tsukuba-shi (JP); Hajime Takeoka, Tokyo (JP)

Correspondence Address: DARBY & DARBY P.C., P.O. BOX 770, Church Street Station, New York, NY, 10008-0770, US

(73) Assignees: Kurume University, Kurume-shi (JP); Kyushu Institute of Technology, Kitakyushu-shi (JP); Sekisui Plastics Co. , Ltd., Osaka-shi (JP); Japan Aerospace Ecploration Agency, Tokyo (JP); Japann Space Forum, Tokyo (JP)

(21) Appl. No.: 12/666,546

(22) PCT Filed: Jun. 25, 2008
(86) PCT No.: PCT/JP2008/061548

§ 371 Date: Mar. 29, 2010

(30) Foreign Application Priority Data
Jun. 27, 2007 (JP) 2007-169554

(51) Int. Cl. A61N 1/04

(52) U.S. Cl. 607/48

(57) ABSTRACT

A garment for electrically stimulating muscles is provided to be used for, e.g., rehabilitation or exercise whereby an electrode can be conveniently provided at such a position as allowing the electrode to stimulate the nerve-muscular junction of a muscle to be moved in toning up the muscle through electrical stimulation on the muscle by taking advantage of the muscular contraction caused by electrically stimulating the muscle. A garment for electrically stimulating muscles comprises a garment body, a required number of openings which are provided in the garment body in such a manner that each opening is located at the nerve-muscular junction or in the vicinity of the nerve-muscular junction of a muscle to be moved in the case where a wearer wears the garment body, and electrodes provided at the respective openings in such a manner that each electrode located on the skin surface at the nerve-muscular junction or in the vicinity of the nerve-muscular junction of a muscle in the case where a wearer wears the garment body.

### CROSS-REFERENCE TO PRIOR APPLICATION

This is the U.S. National Phase Application under 35 U.S.C. §371 of International Patent Application No. PCT/JP2008/061548 filed Jun. 25, 2008, which claims the benefit of Japanese Patent Application No. 2007-169554 filed Jun. 27, 2007, both of which are incorporated in their entirety by reference herein. The International Application was published in Japanese on Dec. 31, 2008 as WO2009/001858 A1 under PCT Article 21(2).

### TECHNICAL FIELD

The present invention relates to a garment for electrically stimulating muscles. More specifically, the present invention relates to a garment used for rehabilitation or exercise, and when electrical muscle stimulation (skeletal muscle stimulation) to train muscles using muscular contraction by electrically stimulating the muscles is conducted, the determination of electrode positions can be easily determined so that the electrodes are set up at a position where the neuromuscular junction of the muscle to be moved can be stimulated.

### BACKGROUND

Electrical muscle stimulation (EMS) has drawn attention as an effective method that can be applied to equipment to assist in rehabilitation and exercise equipment (including so-called health equipment). Exercise based on electrical muscle stimulation differs from exercise using heavy objects such as lifting dumbbells, and has been demonstrated to be a safer exercise method that trains muscles using muscular contraction by electrically stimulating the muscles.

This kind of equipment provides electrodes for electrically stimulating muscles. The positions of the electrodes on the body of the user are normally adjusted or set up based on the experience of the user or of an assistant.

Further, movement using electrical muscle stimulation does not mean that electrical stimulation may stimulate any location or position of the muscle, but rather in order to have a full movement effect it is extremely important to set up the electrodes in positions that can stimulate the neuromuscular junction (part where the motor nerve joins the muscle: motor point) of the muscle.

The muscle strengthener described in JP 3026007 B1 proposed by the present applicant is one example of the aforementioned exercise equipment.

This apparatus for strengthening muscles includes a training harness composed of an electrode unit that electrically stimulates the muscle and causes contraction, a support harness that works together with the training harness, a sensor to detect the change of the angle formed between the training harness and the support harness from a specified angle, and a controller to supply electricity to the electrode unit; and when the sensor has detected, electricity is supplied to the electrode unit to electrically stimulate the muscle that is the main motor muscle or the muscle that is the antagonist muscle.

### SUMMARY

In the general exercise equipment that adopts the method of movement based on electrically stimulating the muscles, it is necessary to search for the exact positions when positioning the electrodes to stimulate the neuromuscular junctures of the muscle. There is the problem that complicated and time consuming operations and manipulations are required when the user or assistant carries out the operations and manipulations to search for these positions based on experience as described above.

Moreover, if a main motor muscle is contracted during the movement by electrical muscle stimulation using the aforementioned apparatus for strengthening muscles of JP 3026007 B1, such apparatus for strengthening muscles can electrically stimulate the antagonist muscle, so that the antagonist muscle causes exercise based on a closed movement chain associated with centrifugal contraction, which is very useful and practical in terms of being able to conduct effective exercise.

Nonetheless, the apparatus for strengthening muscles described above has a structure that provides a training harness containing the electrode unit, and a support harness that works together with it. However, the positions of the electrodes have been already determined on the training harness, and therefore the positions of the electrodes in relation to the body must be determined by the mounting position of the training harness. For this reason it is difficult to make fine adjustments of the electrode positions.

Thus, in an embodiment, the present invention provides a garment for electrically stimulating muscles used, for example, for rehabilitation or exercise equipment, that can simplify the electrode position determination such that the electrodes are set at positions that can stimulate the neuromuscular junctions of the muscles targeted for movement when conducting electrical stimulation of muscles to train muscles utilizing muscle contraction by electrically stimulating the muscle.

The present invention is providing garment for electrically stimulating muscles comprising a garment main body; openings of a specified number provided in the garment main body positioned at the neuromuscular junctions or in the vicinity including the neuromuscular junctions of the muscle targeted for movement when the wearer is wearing the main body of the garment; and electrodes provided in the aforementioned openings positioned on the surface of the skin at the neuromuscular junctions or in the vicinity of the neuromuscular junctions of the muscle targeted for movement when the wearer is wearing the aforementioned garment main body.

Preferably the present invention comprises electrode securing members composed of a non-conductive body that can secure inside the openings the electrodes provided in the openings.

Preferably, the electrode securing member of the present invention is a net member. More preferably the electrode securing member of the present invention is a net member, and the electrode is an adhesive seal electrode that can contact the connection terminal and can adhere to the surface of the skin through the mesh of the aforementioned netting material.

Preferably the present invention provides an electric cord, one end of which is connected to the electrodes and the other end of which can be connected to the electrical muscle stimulation device.

Preferably, the garment main body of the present invention comprises clothing-shaped upper half body garment, lower half body garment, or both.

In order to set the electrodes at accurate positions, that is on the surface of the skin in the vicinity of the neuromuscular junction of the muscle at the time of wearing, more preferably the garment main body is a garment that determines the positions of the various parts of the garment in relation to the body of the wearer by wearing the main body of garment, such as, for example, the upper body garment or lower body garment of a wet suit.

With this kind of garment, even if the positions of the openings and electrodes are previously determined, the inconvenience of discrepancies occurring in the positions of the electrodes in each wearing is less prone to occur.

Moreover, in addition to the so-called clothing structure, as in upper body garment or lower body garment, a structure that is locally mounted on the foot (leg) or hand (arm), for example, a supporter, may be adopted as the garment main body in the present invention.

Preferably, the electrode securing member is provided to cover or close off the opening, but this is not a limit. The electrode securing member can conduct electricity to the skin by allowing electricity conducted through the electrode to be secured to pass from the front surface to the back surface of the electrode securing member, or by connecting the electrode from the front side of the electrode securing member to a conductor provided on part of the back side. Preferably netting (including mesh, a net or the like) provided with this functionality is adopted as the electrode securing member, but this is not a limit, and for example, holes may also be provided in cloth, paper, or sheets or film of a variety of synthetic resins for this electrode securing member.

When an electrode conducts electricity to the skin, preferably the electrode securing member is formed by a non-conductor, but may also be formed by a conductor. If the electrode securing member is formed by a conductor, then all of the electrode securing member that contacts the skin will act as an electrode in the same way as the electrode to be secured, and therefore the size and shape must be adapted for conducting electricity to the skin, specifically, for stimulating the neuromuscular juncture of the muscle, and thus more accurate position determination is required.

The connecting terminals and electrodes are formed using the specified conductive substances, and are not particularly limited by materials, shapes and structures as long as electricity can be conducted to the skin without obstruction. This kind of conductive substance may include, for example, metals such as metal plate, metal wire (including stranded wire), silver, silver-silver chloride compound, nickel, and molybdenum; conductive paste prepared using carbon black or graphite singly or in a mixture of 2 or more kinds; laminates of metal foil such as silver or of metal fibers such as stainless steel fibers; or cloth woven with threads containing metal fibers. Specifically, cloth woven with thread containing metal fibers is flexible and can form to the shape of the body, and therefore is more preferable because this cloth allows contact with the curved surface of the skin without the wearer feeling discomfort.

As long as adhesive seal electrodes have satisfactory adhesion to the surface of the skin, the materials and structure are not particularly limited. For example, an adhesive seal electrode formed by affixing a conductive adhesive tape material, an adhesive seal material, or a conductive adhesive material on a substrate may be adopted. Specifically, an adhesive seal electrode with a structure that includes a substrate sheet, an electro-conductive layer capable of conducting electricity formed using a conductive substance on the surface of the substrate sheet, and a conductive adhesive material layer provided to make contact with this conductive layer is more preferable because the conductive adhesive material layer provides satisfactory adhesion to the surface of the skin, and the contact impedance between the surface of the skin may be reduced since the input area of electricity conducted to the skin can be increased. Moreover, the function of adhesive seal electrode is to enable the electric current that is conducted to the connection terminal unit to pass from the connection terminal through the conductive adhesive material, to spread over the entire surface of the conductive layer formed on the surface of the substrate sheet, and further to be conducted to the surface of the skin through the conductive adhesive material layer.

The aforementioned substrate sheet is not particularly limited, but preferably a flexible resin film or sheet with comparatively high tear strength is adopted in order to allow close adhesion of the conductive adhesive material layer to the surface of the skin. For example, polyethylene terephthalate (PET), polyvinyl chloride, polyethylene (PE), polypropylene (PP) and the like may be adopted as this kind of resin film or sheet, but polyethylene terephthalate, which has high tear strength and is easily printed on, is more preferable. Further, the thickness of the substrate sheet is not particularly limited, but may be suitably set, for example, at 10 to 500μ.

The aforementioned conductive layer formed on the surface of the substrate sheet is not particularly limited as long it can be implemented without interfering with the flow of electricity, and may be formed using the specified conductive material. Examples of this kind of conductive material include metals such as silver, silver-silver chloride compound, nickel, and molybdenum, or conductive pastes prepared using carbon black or graphite singly or in a mixture of 2 or more kinds. Moreover, the electrode unit may be formed by making laminates of metal foil such as silver or of metal fibers such as stainless steel fibers.

The aforementioned conductive adhesive material layer is not particularly limited as long as the layer is conductive, does not irritate the skin excessively, and has sufficient adhesive force. Optimally, a conductive adhesive gel is used in which water and electrolytic salt and the like are added to a matrix of a polyacrylate derivative such as sodium polyacrylate, ester polyacrylate or polyacrylamide, or of a poly-N-vinyl acetamide derivative such as polyvinylpyrrolidone or poly-N-vinyl acetamide, or of polyurethane and the like.

### (Action)

The action of the garment for electrically stimulating muscles related to the present invention will be explained. Further, here the codes given to the various parts in the embodiments to be described later will allow correspondences to the various configurational requirements used in the present invention, but in the same way as the codes used to describe the various claims, these codes are ultimately for the purpose of simplifying the understanding of the content, and the meanings of the configurational parts are not limited to those of the various parts of the embodiments.

By a wearer putting on garment for electrically stimulating muscles (A1, A2), electrodes (8) provided in an opening (2, 2a) are positioned on the surface of the skin at the neuromuscular junction or in the vicinity of the neuromuscular junction of the muscle of the wearer targeted for movement. Then, movement can be induced by electrical muscle stimulation (EMS) by conducting electricity to the skin through the electrode (8) with electrical muscle stimulation equipment.

In this way, because the electrodes (8) are installed beforehand in the aforementioned specified positions of the garment (A1, A2), when electrical muscle stimulation is carried out in order to train the muscles by taking advantage of the fact that electrical stimulation of a muscle contracts that muscle, it is not necessary to adjust the position of the electrodes (8) each time of use, and position determination of the electrodes (8) can be simply conducted just by putting on the garment (A1, A2).

Garment that provides an electrode securing member (3), which is formed by non-conductive material and can secure the electrodes (8) provided in the openings (2, 2a), can adjust the positions for securing the electrodes (8) during manufacture or after manufacture of the garment, and therefore, this type of garment is particularly useful, for example, when the garment is divided (A1, A2) into multiple types corresponding to the body types of wearers.

Further, when the electrode securing member (3) is netting that provides multiple mesh openings, the electrodes (8) can make contact with the surface of the skin through the mesh openings even when the electrode is secured to the top surface side of the netting. Moreover, netting has satisfactory air circulation, and has the advantage that the surface of the skin where the electrode makes contact is less prone to become sweaty.

Garment, in which the electrode securing member is netting (3) and in which the electrodes are adhesive seal electrodes (8) that can make contact with connection terminal (4) and adhere to the surface of the skin through the aforementioned mesh openings of the netting (3), is preferable because the input surface area of the electricity conducted to the skin can be increased, and therefore the contact impedance with the surface of the skin is reduced.

Garment that provide an electric cord (5), of which one end is connected to the terminals (8) and the other end can connect to an electrical muscle stimulation device (C1), enables exercise by electrical muscle stimulation to be easily conducted by connecting the electrical muscle stimulation device (C1) to the electric cord (5).

By the wearer wearing a garment main body (1) configured by clothing-shaped upper body garment, lower body garment, or both, the positions of each part of the garment (A1, A2) are necessarily determined in relation to the body of the wearer so that even if the positions of the openings (2, 2a) and electrodes (8) are determined in advance, the inconvenience of discrepancies occurring in the positions of the electrodes in each wearing is less prone to occur.

The present invention is used, for example, as a garment for rehabilitation and exercise, and when conducting electrical muscle stimulation to train muscles by taking advantage of the fact that electrical stimulation of a muscle contracts that muscle, garment for electrically stimulating muscles can be offered in which electrode positions can be easily determined such that the electrodes are set up in positions to stimulate the neuromuscular junctions of the muscles.

### BRIEF DESCRIPTION OF THE DRAWINGS

FIG. 1 is a front view diagram indicating a first embodiment of garment for electrically stimulating muscles related to present invention;

FIG. 2 is a back view diagram of the garment for electrically stimulating muscles in FIG. 1;

FIG. 3 is an expanded explanatory diagram indicating the assembly structure of the opening with the netting and connecting terminals;

FIG. 4 is an end surface explanatory diagram of the I-I part in FIG. 3;

FIG. 5 is an explanatory diagram indicating the state when an adhesive seal electrode is affixed to the connecting terminal and the netting part;

FIG. 6 is a cross-sectional explanatory diagram indicating the adhesive seal electrode;

FIG. 7 is an explanatory diagram indicating the use state;

FIG. 8 is a front view diagram indicating a second embodiment of garment for electrically stimulating muscles related to present invention; and

FIG. 9 is a back view diagram of the garment for electrically stimulating muscles in

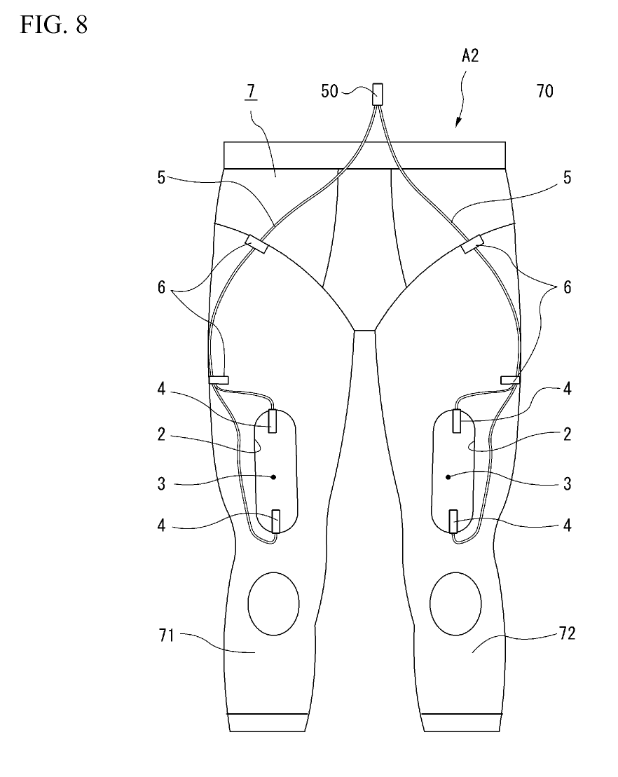

FIG. 8.

### LEGEND

A1 Garment for electrically stimulating muscles
1 Garment main body, 10 Torso part, 11, 12 Sleeve
2, 2a Opening, 20 Piping, 21 Stitching
3 Mesh
4 Connecting terminal, 40 Sewed part
5 Electric cord, 50 terminal
6 Pass-through loop
8 Adhesive seal electrode, 80 Substrate sheet, 81 Conductive layer, 82 Conductive adhesive material
9 Fastener
B Belt
C1 Electrical muscle stimulation device, C2 Controller
M Wearer
A2 Garment for electrically stimulating muscles
7 Garment main body, 70 Hip part, 71 Leg part

### DETAILED DESCRIPTION

The present invention will be explained in detail based on the embodiments indicated in the drawings.

### Embodiment 1

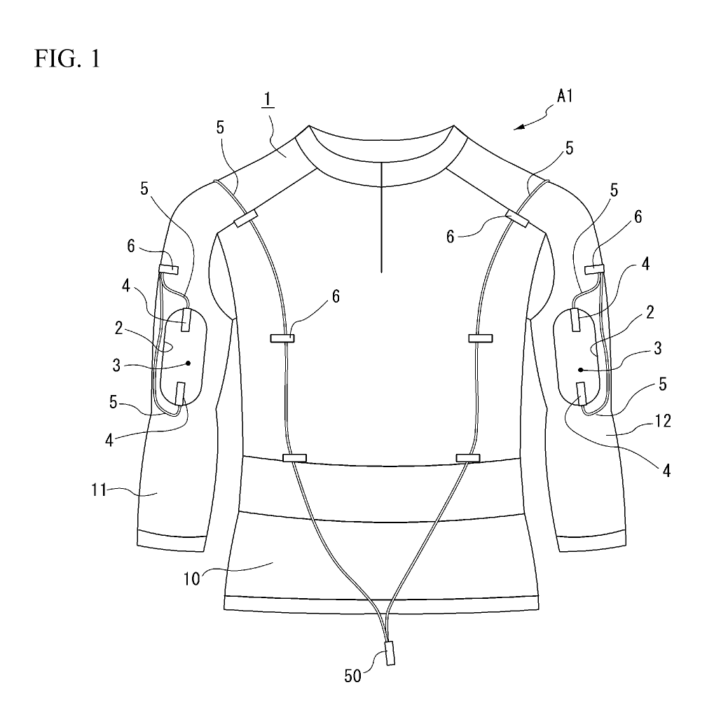

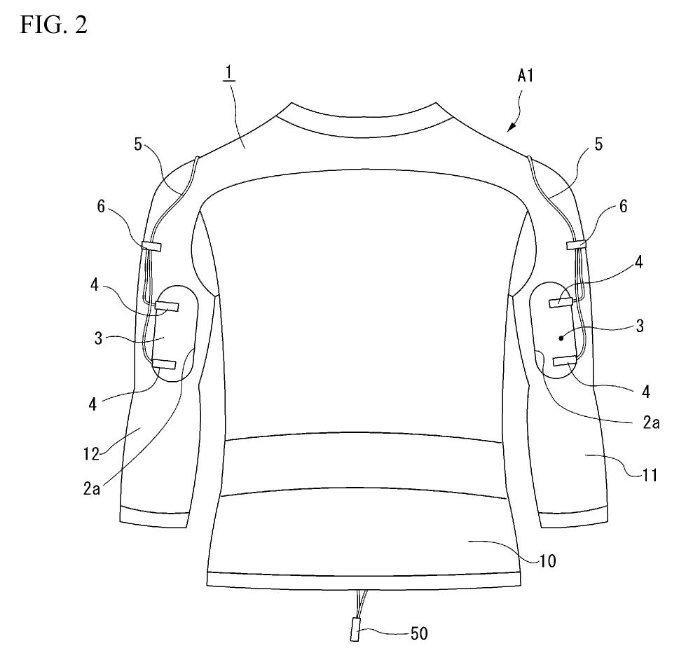

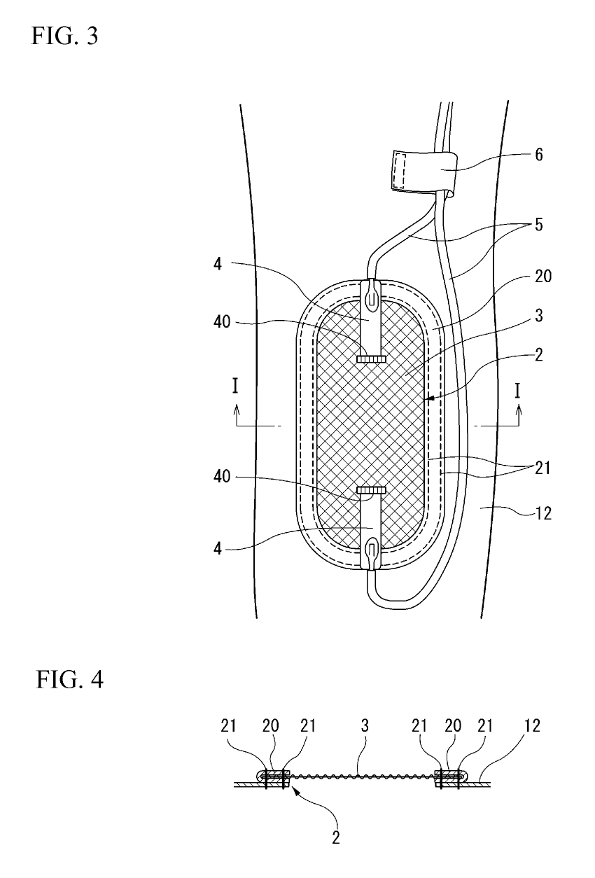

FIG. 1 is a front view diagram indicating a first embodiment of garment for electrically stimulating muscles related to present invention; FIG. 2 is a back view diagram of the garment for electrically stimulating muscles in FIG. 1; FIG. 3 is an expanded explanatory diagram indicating the assembly structure of the opening with the netting and connecting terminals; and FIG. 4 is an end surface explanatory diagram of the I-I part in FIG. 3.

Further, indication of the adhesive seal electrode 8 is omitted from FIG. 1, FIG. 2, FIG. 3, and FIG. 4, respectively. Moreover, indication of the electric cords is omitted from FIG. 4.

The garment for electrically stimulating muscles A1 has a garment main body 1. The garment main body 1 is clothing-shaped garment for the upper body. The garment main body 1 is a non-conductor (insulator), and is formed by cloth having slight elasticity.

Openings 2, 2a have nearly oval edge shapes, and are provided respectively on the front and back sides of the upper arms part of sleeves 11, 12 that are provided on both sides of a torso part 10 of the garment main body 1. Each opening part 2 on the front side is open so as to correspond to the brachial biceps of the upper arm of the wearer when worn, and each opening part 2a on the back side is open so as to correspond to the brachial triceps. Further, the shapes of the openings 2, 2a are not particularly limited to ovals, and a variety of suitable shapes such as circles or squares may be suitably adopted.

A synthetic resin mesh 3 is assembled on the front side edge of the openings 2, 2a (total of four locations) using a cloth piping 20 (refer to FIG. 3). The piping 20 and the fabric of sleeves 11 and 12 are unified by applying stitching 21 following along both sides in the width direction of the piping 20 so that the mesh 3 closes off the openings 2, 2a (air can circulate through visually transparent mesh). Further, the aforementioned material of the piping 20 and the mesh 3 is insulating. The size of the mesh openings of the mesh 3 is not particularly limited as long as the adhesive seal electrodes 8 to be described later can make contact with the surface of the skin of the wearer through the mesh openings.

Connecting terminals 4 are respectively assembled on the front side of the meshes 3 closing off the openings 2, 2a. The connecting terminals 4 are conductive cloth (woven with thread containing metal fiber) formed into rectangles, and insulating sheet (omitted from the diagram) is provided on the entire surface on the side contacting the mesh 3. Connecting terminals 4 are assembled symmetrically in 2 locations on both ends of the long dimensional side over the mesh 3 provided on the opening 2 of the front side (refer to FIG. 1 and FIG. 3). Moreover, connecting terminals 4 are assembled in parallel at a required interval at two edge locations on one end of the short dimensional side over the mesh 3 provided on the opening 2a of the back side (refer to FIG. 2).

The connecting terminals 4 are secured by sewing one lengthwise end onto the piping 20, and the other end is secured to the thread part of the mesh 3 by a sewed part 40. Moreover, one end of the electric cord 5 is connected to the piping side ends of the connecting terminals 4, and the connecting terminals 4 and the garment main body 1 have an insulating relationship (relationship in which electricity is not conducted to the garment main body 1). At the time of use, the adhesive seal electrodes 8 are affixed so as to contact and cover the connecting terminals 4.

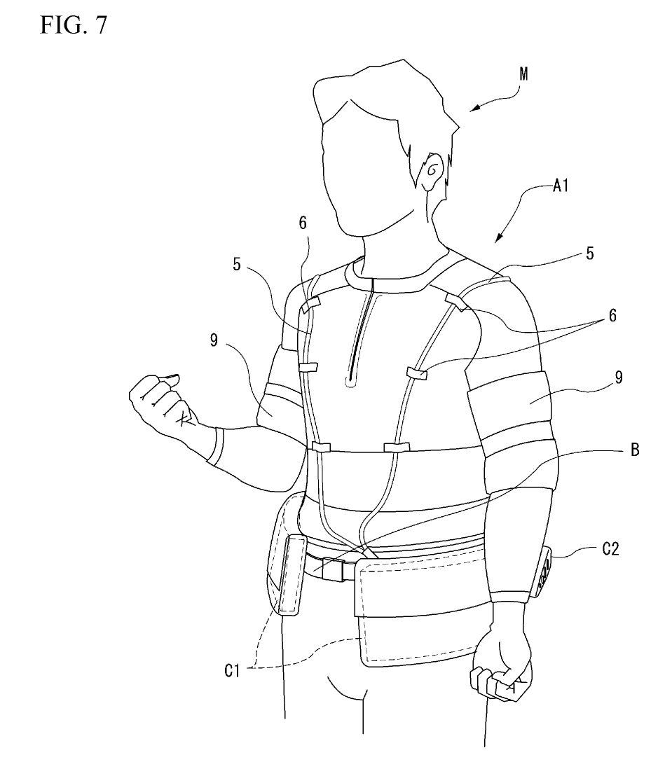

As indicated in FIG. 6 to be described later, the adhesive seal electrodes 8 include in a layered form such that a conductive layer 81 and a conductive adhesive material 82 are provided over the entire surface of one side of a substrate sheet 80 formed by a resin film. Electricity passes through the connecting terminals 4 to the conductive adhesive material 82 of the adhesive seal electrode 8, and the conductive adhesive material 82 can adhere tightly and pass electricity to the surface of the skin through the mesh openings of the mesh 3.

The electric cords 5, which extend from the connecting terminals 4 provided in two locations each of the openings 2, 2a, for a total of 8 locations, turn at both shoulders from the back sides of sleeves 11 and 12 respectively, and then are led to the front side of the torso part 10. The electric cords 5 are bundled at the front side of the torso part 10, and a terminal 50 for connecting to the electrical muscle stimulation device is provided at the end of the cords. Further, the cords 5 pass through pass-through loops 6 provided on the surface of the garment main body 1 at the required locations.

### (Action)

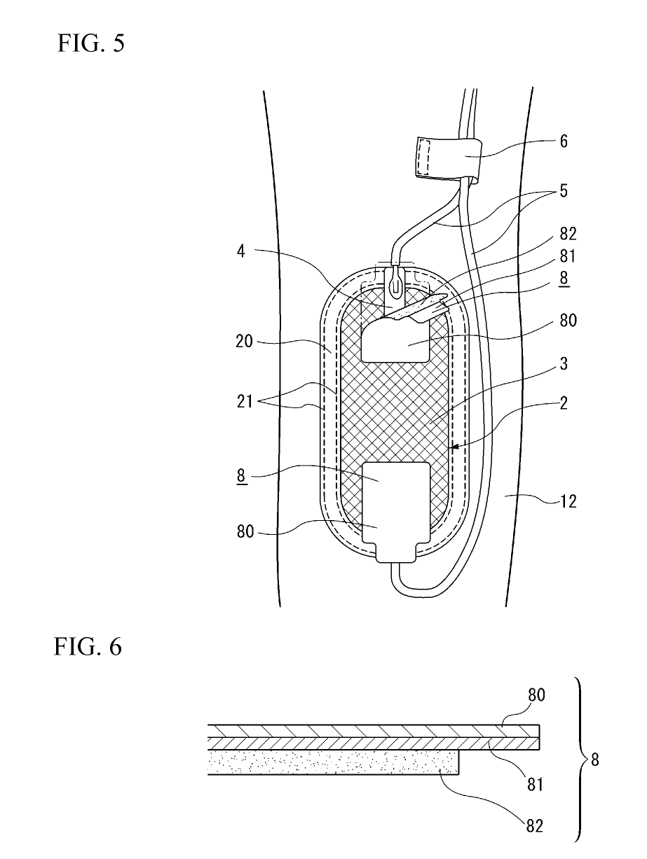

FIG. 5 is an explanatory diagram indicating the state when an adhesive seal electrode is affixed to the connecting terminal and the netting part; FIG. 6 is a cross-sectional explanatory diagram indicating the adhesive seal electrode; and FIG. 7 is an explanatory diagram indicating the use state.

The garment for electrically stimulating muscles A1 is particularly useful for the wearer to exercise the brachial triceps and brachial biceps of both arms. Moreover, by combining the garment with an electrical muscle stimulation device C1 to control electrical stimulation, exercise combining a agonist and the antagonist muscle in a closed movement chain with centrifugal contraction is possible.

By the wearer M wearing the garment for electrically stimulating muscles A1, the terminals 4 set up at the openings 2, 2a are positioned in the proximity of the surface of the skin, with the mesh 3 in between, in the vicinity of the neuromuscular junctures of the muscles (brachial triceps and brachial biceps) to be exercised of the wearer M. Further, the garment for electrically stimulating muscles A1 is clothing-shaped, and the positions of the various parts of the garment main body 1 are necessarily determined in relation to the body of the wearer M, and therefore even if the positions of the openings 2, 2a and the connecting terminals 4 are determined in advance, the inconvenience of discrepancies occurring in the positions of the adhesive seal electrodes 8 affixed to cover the connecting terminals 4 is less prone to occur between each wearing.

Next, as indicated in FIG. 5, the adhesive seal electrodes 8 are affixed to cover the connecting terminals 4. At this time, by firmly pressing the adhesive seal electrodes 8 to the mesh 3 side, the conductive adhesive material 82, which is the adhesive surface of the adhesive seal electrodes 8, adheres to the surface of the skin through the mesh openings of the mesh 3. Electricity can thereby be conducted over a broad surface area in relation to the surface of the skin via the adhesive seal electrodes 8 that are contacting the connecting terminals 4 through the conductive adhesive material 82, and the adhesive seal electrodes 8 can effectively apply electrical stimuli to the neuromuscular junctions (motor points) of the muscles at these positions.

Further, when exercising, a fastener 9 is wound on the upper arm as indicated in FIG. 7, so that contact between the adhesive seal electrodes 8 and the surface of the skin can be more securely supported. Further, the mesh 3 has adequate air ventilation, and there is the advantage that even when the garment is used for a comparatively long time, sweating is not prone to occur on the surface of the skin where the adhesive seal electrode 8 makes contact or on the surface of the skin in that vicinity.

Then, using the electrical muscle stimulation device C1 assembled on the waist of the wearer M with a belt B, exercise can be conducted using electrical muscle stimulation (EMS) by conducting electricity to the surface of the skin through the adhesive seal electrodes 8.

Here, the method of exercise based on electrical stimulation using the garment for electrically stimulating muscles A1 and the electrical muscle stimulation device C1 will be given as a simple explanation.

(1) For Movement that Causes Flexion of the Elbow Joint

In normal movement that causes flexion from the state of having the elbow joint extended, the brachial biceps is taken as the agonist and is contracted, and the brachial triceps is taken as the antagonist muscle and is relaxed. Specifically, when movement is going on in this way, electricity is conducted to the surface of the skin from the adhesive seal electrodes 8 set up at the back side openings 2a of the sleeves 11, 12 of the garment for electrically stimulating muscles A1, there is no conducting of electricity from the other adhesive seal electrodes 8.

Consequently, the brachial triceps is not in the relaxed state, but contract to a suitable degree. Specifically, in this movement, the resistance caused by contraction of the brachial triceps, which originally forms the antagonist muscle, becomes a suitable degree of load and can train the brachial biceps, which is the primary muscle, and by applying a load of centrifugal contraction in relation to the brachial triceps, the brachial triceps can thereby be simultaneously trained.

(2) For Movement that Causes Extension of the Elbow Joint

In normal movement that causes extension from the state of having the elbow joint flexed, the brachial triceps is taken as the agonist and is contracted, and the brachial biceps is taken as the antagonist muscle and is relaxed. Specifically, when movement is going on in this way, electricity is conducted to the surface of the skin from the adhesive seal electrodes 8 set up at the front side openings 2 of the sleeves 11, 12 of the garment for electrically stimulating muscles A1. At this time no electricity is conducted from the other adhesive seal electrodes 8.

Consequently, the brachial biceps is not in the relaxed state, but is contracted. Specifically, in this movement, the resistance caused by contraction of the brachial biceps, which originally forms the antagonist muscle, becomes a suitable degree of load and can train the brachial triceps, which is the primary muscle, and by applying a load of centrifugal contraction in relation to the brachial biceps, the brachial biceps can thereby be simultaneously trained.

Further, a controller C2 for setting up and adjusting control based on the electric muscle stimulation device C1 has been installed on the left wrist of the wearer M.

Moreover, continuous alternating repetition of the aforementioned movements is conducted, and switching of the control at the time of each movement is conducted using various sensors. Details of this kind of controller and the control method will be omitted here. The same will be done in the explanation of the action of the garment for electrically stimulating muscles A2 to be described later.

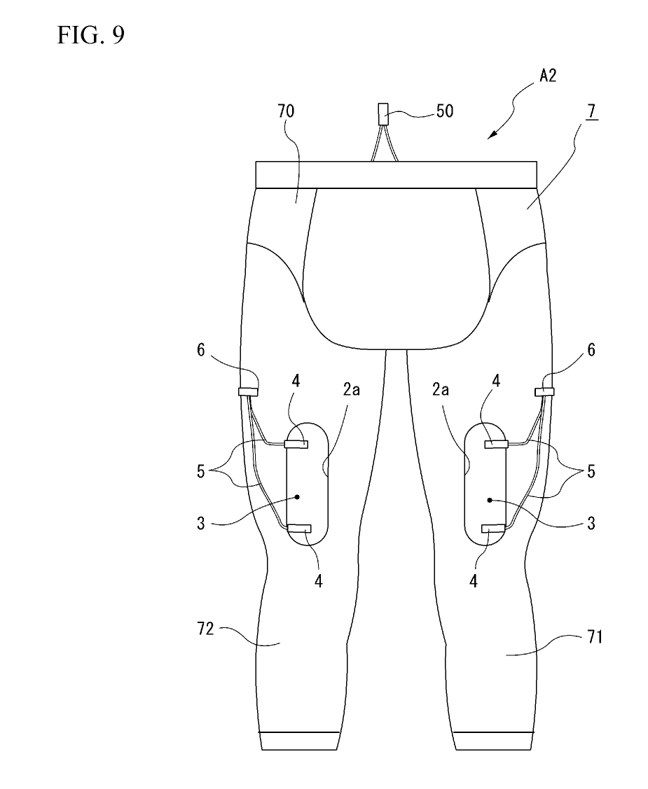

FIG. 8 is a front view diagram indicating a second embodiment of garment for electrically stimulating muscles related to present invention; and FIG. 9 is a back view diagram of the garment for electrically stimulating muscles in FIG. 8.

Further, in the present embodiment, the same codes are attached to locations in the diagrams equivalent to those of the aforementioned garment for electrically stimulating muscles A1, and redundant explanations of the basic structures will be omitted. Moreover, depiction of the adhesive seal electrodes 8 has been omitted in FIG. 8 and FIG. 9.

The garment for electrically stimulating muscles A2 has a garment main body 7. The garment main body 7 is clothing-shaped lower body garment (pants). The garment main body 7 is non-conductive (insulating), and is formed of cloth having slight elasticity.

Openings 2, 2a have nearly oval edge shapes, and are provided respectively on the front and back sides of the thigh part of leg parts 71, 72 that are provided on both sides of a hip part 70 of the garment main body 7. The opening parts 2 on the front side open so as to correspond to the femoral quadriceps of the thigh of the wearer when worn, and opening parts 2a on the back side open so as to correspond to the femoral biceps. Further, the shapes of the openings 2, 2a are not particularly limited to ovals, and a variety of suitable shapes such as circles or squares may be suitably adopted.

The piping 20, mesh 3 and connecting terminals 4 all have the same structures as in the garment for electrically stimulating muscles A1 described above are set up on the side edge surface of the openings 2, 2a (total of four locations). Moreover, one end of the electric cord 5 is connected to the connecting terminals 4 in the same way, and the terminal 50 is set up on the tip of the bundled electric cords 5 in the same way.

### (Action)

A simple explanation will be given of the method of movement based on electrical stimulation using the garment for electrically stimulating muscles A2 and the electrical stimulation device C1.

(1) For Movement that Causes Flexion of the Knee Joint

In normal movement that causes flexion from the state of having the knee joint extended, the femoral biceps is taken as the agonist and is contracted, and the femoral quadriceps is taken as the antagonist muscle and is relaxed. Specifically, when the movement is going on in this way, electricity is conducted to the surface of the skin from the adhesive seal electrodes 8 set up at the front side openings 2 of the leg parts 71, 72 of the garment for electrically stimulating muscles A2, there is no conducting of electricity from the other adhesive seal electrodes 8.

Consequently, the femoral quadriceps is not in the relaxed state, but contracts to be a suitable degree. Specifically, in this movement, the resistance caused by contraction of the femoral quadriceps, which originally forms the antagonist muscle, becomes a suitable degree of load and can train the femoral biceps, which is the agonist, and by applying a load of centrifugal contraction in relation to the femoral quadriceps, the femoral quadriceps can thereby be simultaneously trained.

(2) For Movement that Causes Extension of the Knee Joint

In normal movement that causes extension from the state of having the knee joint flexed, the femoral quadriceps is taken as the agonist and is contracted, and the femoral biceps is taken as the antagonist muscle and is relaxed. Specifically, when the movement is going on in this way, electricity is conducted to the surface of the skin from the adhesive seal electrodes 8 set up at the back side openings 2a of the leg parts 71, 72 of the garment for electrically stimulating muscles A2, there is no conducting of electricity from the other adhesive seal electrodes 8.

Consequently, the femoral biceps is not in the relaxed state, but contracts to be a suitable degree. Specifically, in this movement, the resistance caused by contraction of the femoral biceps, which originally forms the antagonist muscle, becomes a suitable degree of load and can train the femoral quadriceps, which is the agonist, and by applying a load of centrifugal contraction in relation to the femoral biceps, the femoral quadriceps can thereby be simultaneously trained.

Further, the terminology and expressions used in this Description, which were adopted strictly for explanatory purposes, are not limited in any way, and there was no intention to exclude terminology or expressions equivalent in value to all or part of the characteristics described in this Description. In addition, obviously a variety of modifications of the embodiments is possible within the scope of the technical concepts of the present invention.

According to the present invention, the garment for electrically stimulating muscles is used, for example, for rehabilitation or exercise equipment, in which determination of electrode positioning can be easily implemented when conducting electrical muscle stimulation to train muscles utilizing the contraction of muscles by electrical stimulation of the muscles, such that the electrodes can be set up at positions that can stimulate the neuromuscular junctions of the muscle to be exercised.

1. Garment for electrically stimulating muscles comprising:
a garment main body;
a predetermined number of openings, which are disposed in the garment main body so as to be positioned at the neuromuscular junction or in the vicinity including the neuromuscular juncture of the muscle to be exercised when a wearer is wearing said garment main body; and
electrodes which are disposed in said openings so as to be positioned at the surface of the skin at the neuromuscular junction or in the vicinity of the neuromuscular juncture of the muscle to be exercised when a wearer is wearing said garment main body.

2. The garment for electrically stimulating muscles according to claim 1, further comprising:
electrode securing members, which are formed in a non-conductive body and configured to secure the electrodes in the openings at inner positions of the openings.

3. The garment for electrically stimulating muscles according to claim 2, wherein the electrode securing members are a mesh.

4. The garment for electrically stimulating muscles according to claim 2, wherein the electrode securing members are a mesh, and the electrodes are adhesive seal electrodes that are configured to contact connecting terminals and to adhere to the surface of the skin through a mesh opening of said mesh.

5. The garment for electrically stimulating muscles according to claim 1, further comprising an electric cord, of which one end is connected to the electrodes and the other end can be connected to an electrical muscle stimulation device.

6. The garment for electrically stimulating muscles according to claim 1, wherein the garment main body comprises at least one of a clothing-shaped upper body garment and a lower body garment.
